(19) United States
(12) Patent Application Publication
(10) Pub. No.: US 2010/0030063 A1
(43) Pub. Date: Feb. 4, 2010

(54) SYSTEM AND METHOD FOR TRACKING AN INSTRUMENT

(75) Inventors: Nathan Tyler Lee, Golden Valley, MN (US); Rick Dean McVenes, Isanti, MN (US); Can Cinbis, Shoreview, MN (US); Jonathan Leslie Kuhn, Ham Lake, MN (US); James F. Kelley, Coon Rapids, MN (US)

Correspondence Address: HARNESS, DICKEY & PIERCE, P.L.C., P.O. BOX 828, BLOOMFIELD HILLS, MI, 48303, US

(73) Assignee: Medtronic, Inc., Minneapolis, MN (US)

(21) Appl. No.: 12/183,674

(22) Filed: Jul. 31, 2008

(51) Int. Cl. A61B 5/05

(52) U.S. Cl. 600/424

(57) ABSTRACT

A system for tracking an instrument relative to an anatomical structure is provided. The system can include at least one tracking device, which can be coupled to the instrument. The system can also include a shape sensor coupled to the instrument that can determine a shape of the instrument. The system can include a tracking system that can track a position of the at least one tracking device relative to the anatomical structure. The system can further include a navigation system that can determine a position and shape of the instrument relative to the anatomical structure based on the position of the at least one tracking device determined by the tracking system and the shape of the instrument as sensed by the shape sensor.

### FIELD

The present disclosure relates generally to navigated surgery, and more specifically, to systems and methods for tracking an instrument, such as an elongated flexible body.

### BACKGROUND

The statements in this section merely provide background information related to the present disclosure and may not constitute prior art.

Image guided medical and surgical procedures utilize patient images (image data) obtained prior to or during a medical procedure to guide a physician performing the procedure. Recent advances in imaging technology, especially in imaging technologies that produce highly-detailed, two, three, and four dimensional images, such as computed tomography (CT), magnetic resonance imaging (MRI), fluoroscopic imaging (such as with a C-arm device), positron emission tomography (PET), and ultrasound imaging (US) has increased the interest in navigated medical procedures.

Generally, during a navigated procedure, images are acquired by a suitable imaging device for display on a workstation. The navigation system tracks the patient, instruments and other devices in the surgical field or patient space. These tracked devices are then displayed relative to the image data on the workstation in image space. In order to track the patient, instruments and other devices, the patient, instruments and other devices can be equipped with tracking devices.

Typically, tracking devices are coupled to an exterior surface of the instrument, and can provide the surgeon, via the tracking system, an accurate depiction of the location of that instrument in the patient space. In cases where the instrument is an elongated flexible body for insertion into an anatomical structure, it may be difficult to determine the shape of the instrument within the anatomical structure.

### SUMMARY

A system for tracking an instrument relative to an anatomical structure is provided. The system can include at least one tracking device, which can be coupled to the instrument. The system can also include a shape sensor coupled to the instrument that can determine a shape of the instrument. The system can include a tracking system that can track a position of the at least one tracking device relative to the anatomical structure. The system can further include a navigation system that can determine a position and shape of the instrument relative to the anatomical structure based on the position of the at least one tracking device determined by the tracking system and the shape of the instrument as sensed by the shape sensor.

Further provided is a method for tracking an instrument relative to an anatomical structure. The method can include positioning at least one tracking device on the instrument, coupling a shape sensor to the instrument and tracking the at least one tracking device relative to the anatomical structure. The method can also include sensing a shape of the instrument, and determining, based on the tracking of the at least one tracking device and the shape of the instrument, a position of instrument relative to the anatomical structure. The method can also include displaying the position of the instrument and the shape of the instrument relative to the anatomical structure as an icon superimposed on an image of the anatomical structure.

Also provided is a system for tracking an instrument relative to an anatomical structure. The system can include an elongated flexible body, which can have a proximal end and a distal end for insertion into the anatomical structure. The system can also include at least one tracking device, which can be coupled to the proximal end, the distal end, a portion of the elongated flexible body between the proximal end and the distal end or combinations thereof. The system can include at least one optical fiber coupled to the elongated flexible body that includes a plurality of strain sensors, and a tracking system that can track a position of the tracking device relative to the anatomical structure. The system can further include an optical system that can read the plurality of strain sensors on the at least one optical fiber. The system can include a navigation system that can determine a position of the elongated flexible body based on the tracking of the first tracking device and a shape of the elongated flexible body based on the reading of the plurality of strain sensors. The system can also include a display that can display an image of the anatomical structure with the position and shape of the elongated flexible body superimposed on the anatomical structure.

Further areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way.

FIG. 1 is a diagram of a navigation system for performing a surgical procedure on a patient according to various embodiments of the present disclosure;

FIG. 2 is a simplified schematic illustration of the patient of FIG. 1, including an instrument according to various embodiments of the present disclosure;

FIG. 2A is a schematic illustration of a portion of the instrument of FIG. 2;

FIG. 3 is a simplified schematic illustration of the patient of FIG. 2, including the instrument according to one of various embodiments of the present disclosure;

FIG. 4 is a simplified schematic illustration of the patient of FIG. 2, including the instrument according to one of various embodiments of the present disclosure;

FIG. 5 is a schematic illustration of a portion of the instrument according to one of various embodiments of the present disclosure;

FIG. 6 is a simplified block diagram illustrating the navigation system of FIG. 1;

FIG. 7 is a graphical representation of an exemplary display produced by the navigation system of FIG. 1;

FIG. 8 is a graphical representation of an exemplary display produced by the navigation system of FIG. 1;

FIG. 9 is a graphical representation of an exemplary display produced by the navigation system of FIG. 1;

FIG. 10 is a dataflow diagram illustrating a control system performed by a control module associated with the navigation system of FIG. 1; and

FIG. 11 is a flowchart illustrating a control method performed by the control module.

### DETAILED DESCRIPTION

The following description is merely exemplary in nature and is not intended to limit the present disclosure, application, or uses. It should be understood that throughout the drawings, corresponding reference numerals indicate like or corresponding parts and features. As indicated above, the present teachings are directed toward providing a system and method for tracking an instrument for use in a navigated surgical procedure. It should be noted, however, that the present teachings could be applicable to any appropriate procedure in which it is desirable to determine a shape of an elongated body within a structure in which the elongated body is flexible and hidden from view. Further, as used herein, the term “module” can refer to an application specific integrated circuit (ASIC), an electronic circuit, a processor (shared, dedicated, or group) and memory that executes one or more software or firmware programs, a combinational logic circuit, and/or other suitable software, firmware programs or components that provide the described functionality. Therefore, it will be understood that the following discussions are not intended to limit the scope of the appended claims.

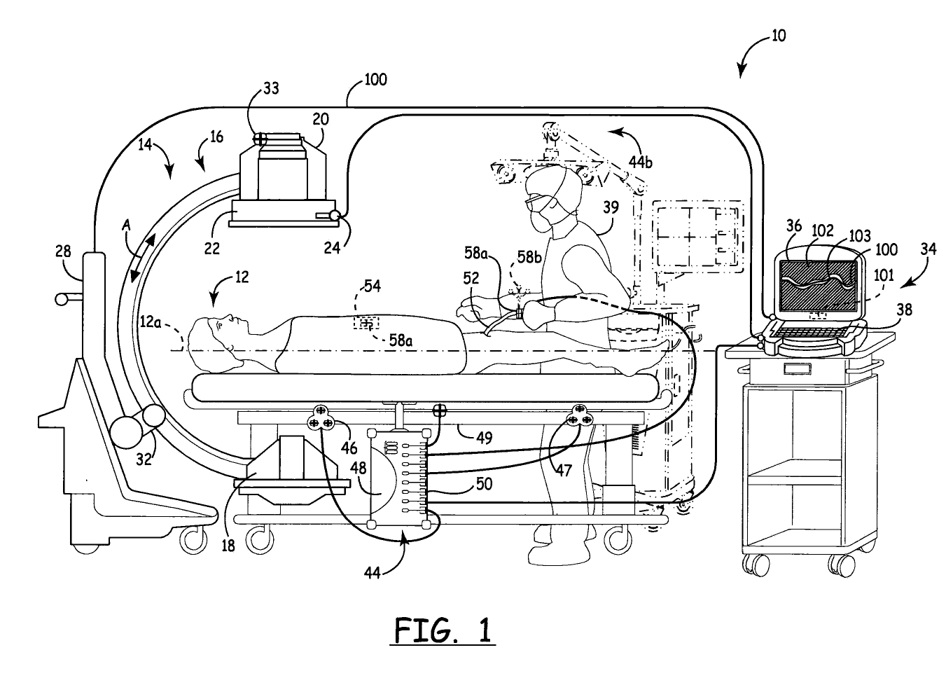

FIG. 1 is a diagram illustrating an overview of a navigation system 10 that can be used for various procedures. The navigation system 10 can be used to track the location of an implant, such as a spinal implant or orthopedic implant, relative to a patient 12. Also the navigation system 10 can track the position and orientation of various instruments. It should further be noted that the navigation system 10 may be used to navigate any type of instrument, implant, or delivery system, including: guide wires, arthroscopic systems, cardiac leads, orthopedic implants, spinal implants, deep-brain stimulator (DBS) probes, etc. Moreover, these instruments may be used to navigate or map any region of the body. The navigation system 10 and the various instruments may be used in any appropriate procedure, such as one that is generally minimally invasive, arthroscopic, percutaneous, stereotactic, or an open procedure.

The navigation system 10 may include an imaging device 14 that is used to acquire pre-, intra-, or post-operative or real-time image data of a patient 12. Alternatively, various imageless systems can be used or images from atlas models can be used to produce patient images, such as those disclosed in U.S. Patent Pub. No. 2005-0085714, filed Oct. 16, 2003, entitled “Method And Apparatus For Surgical Navigation Of A Multiple Piece Construct For Implantation,” incorporated herein by reference. The imaging device 14 can be, for example, a fluoroscopic x-ray imaging device that may be configured as an O-arm™ or a C-arm 16 having an x-ray source 18, an x-ray receiving section 20, an optional calibration and tracking target 22 and optional radiation sensors 24. It will be understood, however, that patient image data can also be acquired using other imaging devices, such as those discussed above and herein.

In operation, the imaging device 14 generates x-rays from the x-ray source 18 that propagate through the patient 12 and calibration and/or tracking target 22, into the x-ray receiving section 20. This allows real-time visualization of the patient 12 and radio-opaque instruments, via the X-rays. In the example of FIG. 1, a longitudinal axis 12a of the patient 12 is substantially in line with a mechanical rotational axis 32 of the C-arm 16. This can enable the C-arm 16 to be rotated relative to the patient 12, allowing images of the patient 12 to be taken from multiple directions or about multiple planes. An example of a fluoroscopic C-arm X-ray device that may be used as the optional imaging device 14 is the “Series 9600 Mobile Digital Imaging System,” from GE Healthcare (formerly OEC Medical Systems, Inc.) of Salt Lake City, Utah. Other exemplary fluoroscopes include bi-plane fluoroscopic systems, ceiling fluoroscopic systems, cath-lab fluoroscopic systems, fixed C-arm fluoroscopic systems, isocentric C-arm fluoroscopic systems, 3D fluoroscopic systems, etc. An exemplary O-arm™ imaging device is available from Medtronic Navigation, Inc. of Littleton, Mass.

When the x-ray source 18 generates the x-rays that propagate to the x-ray receiving section 20, the radiation sensors 24 can sense the presence of radiation, which is forwarded to an imaging device controller 28, to identify whether or not the imaging device 14 is actively imaging. This information can also be transmitted to a coil array controller 48, further discussed herein.

The imaging device controller 28 can capture the x-ray images received at the x-ray receiving section 20 and store the images for later use. Multiple two-dimensional images taken by the imaging device 14 may also be captured and assembled by the imaging device controller 28 to provide a larger view or image of a whole region of the patient 12, as opposed to being directed to only a portion of a region of the patient 12. For example, multiple image data of a leg of the patient 12 may be appended together to provide a full view or complete set of image data of the leg that can be later used to follow contrast agent, such as Bolus tracking. The imaging device controller 28 may also be separate from the C-arm 16 and/or control the rotation of the C-arm 16. For example, the C-arm 16 can move in the direction of arrow A or rotate about the longitudinal axis 12a of the patient 12, allowing anterior or lateral views of the patient 12 to be imaged. Each of these movements involves rotation about a mechanical rotational axis 32 of the C-arm 16. The movements of the imaging device 14, such as the C-arm 16 can be tracked with a tracking device 33.

While the imaging device 14 is shown in FIG. 1 as a C-arm 16, any other alternative 2D, 3D or 4D imaging modality may also be used. For example, any 2D, 3D or 4D imaging device, such as an O-arm™ imaging device, isocentric fluoroscopy, bi-plane fluoroscopy, ultrasound, computed tomography (CT), multi-slice computed tomography (MSCT), magnetic resonance imaging (MRI), high frequency ultrasound (HFU), positron emission tomography (PET), optical coherence tomography (OCT), intra-vascular ultrasound (IVUS), ultrasound, intra-operative CT or MRI may also be used to acquire 2D, 3D or 4D pre- or post-operative and/or real-time images or patient image data 100 of the patient 12. For example, an intra-operative MRI system, may be used such as the PoleStar® MRI system sold by Medtronic, Inc.

In addition, image datasets from hybrid modalities, such as positron emission tomography (PET) combined with CT, or single photon emission computer tomography (SPECT) combined with CT, could also provide functional image data superimposed onto anatomical data to be used to confidently reach target sites within the patient 12. It should further be noted that the imaging device 14, as shown in FIG. 1, provides a virtual bi-plane image using a single-head C-arm fluoroscope as the imaging device 14 by simply rotating the C-arm 16 about at least two planes, which could be orthogonal planes, to generate two-dimensional images that can be converted to three-dimensional volumetric images. By acquiring images in more than one plane, an icon 103 representing the location of an instrument 52, such as an impacter, stylet, reamer driver, taps, drill, deep-brain stimulator (DBS) probes, cardiac leads, catheter, balloon catheter, basket catheter, or other instrument, or implantable devices introduced and advanced in the patient 12, may be superimposed in more than one view and included in image data 102 displayed on a display 36, as will be discussed.

If the imaging device 14 is employed, patient image data 100 can be forwarded from the imaging device controller 28 to a navigation computer and/or processor or workstation 34. It will also be understood that the patient image data 100 is not necessarily first retained in the imaging device controller 28, but may also be directly transmitted to the workstation 34. The workstation 34 can include the display 36, a user input device 38 and a control module 101. The workstation 34 can also include or be connected to an image processor, navigation processor, and memory to hold instruction and data. The workstation 34 can provide facilities for displaying the patient image data 100 as an image on the display 36, saving, digitally manipulating, or printing a hard copy image of the received patient image data 100.

The user input device 38 can comprise any device that can enable a user to interface with the workstation 34, such as a touchpad, touch pen, touch screen, keyboard, mouse, wireless mouse, or a combination thereof. The user input device 38 allows a physician or user 39 to provide inputs to control the imaging device 14, via the imaging device controller 28, adjust the display settings of the display 36, or control a tracking system 44, as further discussed herein.

The control module 101 can determine the location of a tracking device 58 with respect to the patient space, and can determine a position of the instrument 52 in the patient space. The control module 101 can also determine a shape of the instrument 52 relative to the patient space, and can output image data 102 to the display 36. The image data 102 can include the icon 103 that provides an indication of a location of the instrument 52 with respect to the patient space, illustrated on the patient image data 100, as will be discussed herein.

With continuing reference to FIG. 1, the navigation system 10 can further include the electromagnetic navigation or tracking system 44 that includes a localizer, such as a first coil array 46 and/or second coil array 47, the coil array controller 48, a navigation probe interface 50, a device or instrument 52, a patient tracker or first reference frame or dynamic reference frame (DRF) 54 and one or more tracking devices 58. Other tracking systems can include an optical tracking system 44b, for example the StealthStation® Treon® and the StealthStation® Tria® both sold by Medtronic Navigation, Inc. Further, other tracking systems can be used that include acoustic, radiation, radar, infrared, etc., or hybrid systems such as a system that includes components of both an electromagnetic and optical tracking system, etc. Moreover, a position sensing unit could be employed to determine a position of the instrument 52 relative to the anatomy. An exemplary position sensing unit can comprise the LocaLisa® Intracardiac Navigation System, which is sold by Medtronic, Inc. of Minneapolis, Minn. Additionally, the position sensing unit could comprise the position sensing unit described in U.S. patent Ser. No. 12/117,537, entitled “Method and Apparatus for Mapping a Structure,” incorporated herein by reference in its entirety, or the position sensing unit described in U.S. patent Ser. No. 12/117,549, entitled “Method and Apparatus for Mapping a Structure,” incorporated herein by reference in its entirety. In the case of an electromagnetic tracking system 44, the instrument 52 and the DRF 54 can each include tracking device(s) 58.

The tracking device 58 or any appropriate tracking device as discussed herein, can include both a sensor, a transmitter, or combinations thereof and can be indicated by the reference numeral 58. Further, the tracking device 58 can be wired or wireless to provide a signal or emitter or receive a signal from a system. For example, an electromagnetic tracking device 58a can include one or more electromagnetic coil, such as a tri-axial coil, to sense a field produced by the localizing coil array 46 or 47. One will understand that the tracking device(s) 58 can receive a signal, transmit a signal, or combinations thereof to provide information to the navigation system 10, which can be used to determine a location of the tracking device 58. The navigation system 10 can determine a position of the instrument 52 and the DRF 54 based on the location of the tracking device(s) 58 to allow for accurate navigation relative to the patient 12 in the patient space.

With regard to the optical localizer or tracking system 44b, the optical tracking system 44b can transmit and receive an optical signal, or combinations thereof. An optical tracking device 58b can be interconnected with the instrument 52, or other devices such as the DRF 54. As generally known, the optical tracking device 58b can reflect, transmit or receive an optical signal to/from the optical localizer or tracking system 44b that can be used in the navigation system 10 to navigate or track various elements. Therefore, one skilled in the art will understand, that the tracking device(s) 58 can be any appropriate tracking device to work with any one or multiple tracking systems.

The coil arrays 46, 47 can transmit signals that are received by the tracking device(s) 58. The tracking device(s) 58 can then transmit or receive signals based upon the transmitted or received signals from or to the coil arrays 46, 47. The coil arrays 46, 47 are shown attached to the operating table 49. It should be noted, however, that the coil arrays 46, 47 can also be positioned at any other location, as well and can also be positioned in the items being navigated. The coil arrays 46, 47 include a plurality of coils that are each operable to generate distinct electromagnetic fields into the navigation region of the patient 12, which is sometimes referred to as patient space. Representative electromagnetic systems are set forth in U.S. Pat. No. 5,913,820, entitled “Position Location System,” issued Jun. 22, 1999 and U.S. Pat. No. 5,592,939, entitled “Method and System for Navigating a Catheter Probe,” issued Jan. 14, 1997, each of which are hereby incorporated by reference. In addition, representative electromagnetic systems can include the AXIEM™ electromagnetic tracking system sold by Medtronic Navigation, Inc.

The coil arrays 46, 47 can be controlled or driven by the coil array controller 48. The coil array controller 48 can drive each coil in the coil arrays 46, 47 in a time division multiplex or a frequency division multiplex manner. In this regard, each coil can be driven separately at a distinct time or all of the coils can be driven simultaneously with each being driven by a different frequency. Upon driving the coils in the coil arrays 46, 47 with the coil array controller 48, electromagnetic fields are generated within the patient 12 in the area where the medical procedure is being performed, which is again sometimes referred to as patient space. The electromagnetic fields generated in the patient space induce currents in a tracking device(s) 58 positioned on or in the instrument 52 and DRF 54. These induced signals from the instrument 52 and DRF 54 are delivered to the navigation probe interface 50 and can be subsequently forwarded to the coil array controller 48.

In addition, the navigation system 10 can include a gating device or an ECG or electrocardiogram triggering device, which is attached to the patient 12, via skin electrodes, and in communication with the coil array controller 48. Respiration and cardiac motion can cause movement of cardiac structures relative to the instrument 52, even when the instrument 52 has not been moved. Therefore, patient image data 100 can be acquired from the imaging device 14 based on a time-gated basis triggered by a physiological signal or a physiological event. For example, the ECG or EGM signal may be acquired from the skin electrodes or from a sensing electrode included on the instrument 52 or from a separate reference probe (not shown). A characteristic of this signal, such as an R-wave peak or P-wave peak associated with ventricular or atrial depolarization, respectively, may be used as a reference of a triggering physiological event for the coil array controller 48 to drive the coils in the coil arrays 46, 47. This reference of a triggering physiological event may also be used to gate or trigger image acquisition during the imaging phase with the imaging device 14. By time-gating the image data 102 and/or the navigation data, the icon 103 of the location of the instrument 52 in image space relative to the patient space at the same point in the cardiac cycle may be displayed on the display 36. Further detail regarding the time-gating of the image data and/or navigation data can be found in U.S. Patent Pub. Application No. 2004-0097806, entitled “Navigation System for Cardiac Therapies,” filed Nov. 19, 2002, which is hereby incorporated by reference.

The navigation probe interface 50 may provide the necessary electrical isolation for the navigation system 10. The navigation probe interface 50 can also include amplifiers, filters and buffers to directly interface with the tracking device(s) 58 in the instrument 52 and DRF 54. Alternatively, the tracking device(s) 58, or any other appropriate portion, may employ a wireless communications channel, such as that disclosed in U.S. Pat. No. 6,474,341, entitled “Surgical Communication Power System,” issued Nov. 5, 2002, herein incorporated by reference, as opposed to being coupled directly to the navigation probe interface 50.

The instrument 52 may be any appropriate instrument, such as an instrument for preparing a portion of the patient 12, an instrument for treating a portion of the patient 12 or an instrument for positioning an implant, as will be discussed herein. The DRF 54 of the tracking system 44 can be coupled to the navigation probe interface 50. The DRF 54 may be coupled to a first portion of the anatomical structure of the patient 12 adjacent to the region being navigated so that any movement of the patient 12 is detected as relative motion between the coil arrays 46, 47 and the DRF 54. For example, the DRF 54 can be adhesively coupled to the patient 12, however, the DRF 54 could also be mechanically coupled to the patient 12, if desired. The DRF 54 may include any appropriate tracking device(s) 58 used by the navigation system 10. Therefore, the DRF 54 can include an optical tracking device or acoustic, etc. If the DRF 54 is used with an electromagnetic tracking device 58a, it can be configured as a pair of orthogonally oriented coils, each having the same centerline or may be configured in any other non-coaxial or co-axial coil configurations, such as a tri-axial coil configuration (not specifically shown).

Briefly, the navigation system 10 operates as follows. The navigation system 10 creates a translation map between all points in the radiological image generated from the imaging device 14 in image space and the corresponding points in the anatomical structure of the patient 12 in patient space. After this map is established, whenever a tracked instrument, such as the instrument 52 is used, the workstation 34 in combination with the coil array controller 48 and the imaging device controller 28 uses the translation map to identify the corresponding point on the pre-acquired image or atlas model, which is displayed on display 36. This identification is known as navigation or localization. The icon 103 representing the localized point or instruments 52 can be shown as image data 102 on the display 36.

To enable navigation, the navigation system 10 must be able to detect both the position of the anatomical structure of the patient 12 and the position of the instrument 52. Knowing the location of these two items allows the navigation system 10 to compute and display the position of the instrument 52 in relation to the patient 12 on the display 36. The tracking system 44 can be employed to track the instrument 52 and the anatomical structure simultaneously.

The tracking system 44, if using an electromagnetic tracking assembly, essentially works by positioning the coil arrays 46, 47 adjacent to the patient space to generate a low-energy electromagnetic field generally referred to as a navigation field. Because every point in the navigation field or patient space is associated with a unique field strength, the tracking system 44 can determine the position of the instrument 52 by measuring the field strength at the tracking device 58 location. The DRF 54 can be fixed to the patient 12 to identify a location of the patient 12 in the navigation field. The tracking system 44 can continuously recompute the relative position of the DRF 54 and the instrument 52 during localization and relate this spatial information to patient registration data to enable image guidance of the instrument 52 within and/or relative to the patient 12.

Patient registration is the process of determining how to correlate the position of the instrument 52 relative to the patient 12 to the position on the diagnostic or pre-acquired images. To register the patient 12, a physician or user 39 may use point registration by selecting and storing particular points from the pre-acquired images and then touching the corresponding points on the anatomical structure of the patient 12 with a pointer probe. The navigation system 10 analyzes the relationship between the two sets of points that are selected and computes a match, which correlates every point in the patient image data 100 with its corresponding point on the anatomical structure of the patient 12 or the patient space, as discussed herein. The points that are selected to perform registration are the fiducial markers, such as anatomical landmarks. Again, the landmarks or fiducial markers are identifiable on the images and identifiable and accessible on the patient 12. The fiducial markers can be artificial markers that are positioned on the patient 12 or anatomical landmarks that can be easily identified in the patient image data 100. The artificial landmarks, such as the fiducial markers, can also form part of the DRF 54, such as those disclosed in U.S. Pat. No. 6,381,485, entitled “Registration of Human Anatomy Integrated for Electromagnetic Localization,” issued Apr. 30, 2002, herein incorporated by reference.

The navigation system 10 may also perform registration using anatomic surface information or path information as is known in the art. The navigation system 10 may also perform 2D to 3D registration by utilizing the acquired 2D images to register 3D volume images by use of contour algorithms, point algorithms or density comparison algorithms, as is known in the art. An exemplary 2D to 3D registration procedure, is set forth in U.S. patent Ser. No. 10/644,680, entitled “Method and Apparatus for Performing 2D to 3D Registration,” filed on Aug. 20, 2003, hereby incorporated by reference.

In order to maintain registration accuracy, the navigation system 10 continuously tracks the position of the patient 12 during registration and navigation. This is because the patient 12, DRF 54 and coil arrays 46, 47 may all move with respect to one another during the procedure, even when this movement is not desired. Alternatively the patient 12 may be held immobile once the registration has occurred, such as with a head frame (not shown). Therefore, if the navigation system 10 did not track the position of the patient 12 or area of the anatomical structure, any patient movement after image acquisition would result in inaccurate navigation within that image. The DRF 54 allows the tracking system 44 to register and track the anatomical structure. Because the DRF 54 can be coupled to the patient 12, any movement of the anatomical structure of the patient 12 or the coil arrays 46, 47 can be detected as the relative motion between the coil arrays 46, 47 and the DRF 54. Both the relative motion of the coil arrays 46, 47 and the DRF 54 can be communicated to the coil array controller 48, via the navigation probe interface 50, which can update the registration correlation to thereby maintain accurate navigation.

The navigation system 10 can be used according to any appropriate method or system. For example, pre-acquired images, atlas or 3D models may be registered relative to the patient 12 and the patient space. Generally, the navigation system 10 allows the images on the display 36 to be registered and to accurately display the real time location of the various instruments, such as the instrument 52, and other appropriate items, such as DRF 54. In addition, the DRF 54 may be used to ensure that any planned or unplanned movement of the patient 12 or the coil arrays 46, 47 can be determined and used to correct the image data 102 on the display 36.

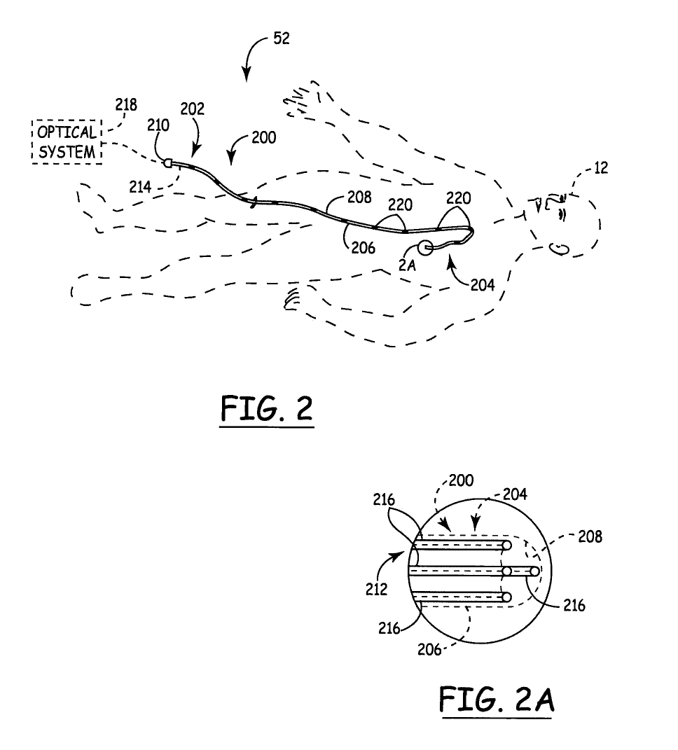

Referring now to FIGS. 1, 2 and 2A, an instrument 52 is shown for use with the tracking system 44. In this case, the instrument 52 comprises an elongated flexible body 200. The elongated flexible body 200 can comprise any suitable generally elongated flexible instrument 52, such as, a catheter, a basket catheter, a balloon catheter, a cardiac lead, guidewire, sheath, endoscope, ablation catheter, arthroscopic instruments, orthopedic instruments, spinal instruments, trocars, deep-brain stimulator (DBS) probes, drug delivery instruments, mapping catheter, etc. As the elongated flexible body 200 can comprise any suitable elongated flexible body, it will be understood that the illustration of the elongated flexible body 200 as a catheter is merely exemplary. Generally, the elongated flexible body 200 can include a proximal end 202, a distal end 204, an exterior surface 206, an interior surface 208, a tracking device 210 and a shape sensor or shape sensing means 212.

The proximal end 202 of the elongated flexible body 200 can generally extend outside of the anatomical structure of the patient 12 when the elongated flexible body 200 is used during the surgical procedure. In some cases, the proximal end 202 can include a graspable portion, generally indicated as 214, to enable the physician or user to manipulate or direct the movement of the distal end 204 of the elongated flexible body 200 within the anatomical structure.

The distal end 204 can comprise a treatment end for treating the anatomical structure. The exterior surface 206 can be configured to be received within the anatomical structure. The exterior surface 206 can be composed of one or more layers of material, and the tracking device 210 and/or the shape sensing means 212 can be coupled to the exterior surface 206, as will be discussed. The interior surface 208 can be configured to enable instruments 52 to pass through the elongated flexible body 200, or could be configured to enable treatment devices or fluids to be directed to the distal end 204. In addition, the tracking device 210 and/or the shape sensing means 212 can be coupled to the interior surface 208, as will be discussed.

The tracking device 210 can comprise any suitable tracking device 58 that can be tracked by the tracking system 44, such as the electromagnetic tracking device 58a or the optical tracking device 58b, however, it should be understood that that tracking device 58 could comprise any suitable device capable of indicating a position and/or orientation of the elongated flexible body 200, such as electrodes responsive to a position sensing unit, for example, the LocaLisa® Intracardiac Navigation System, provided by Medtronic, Inc. In addition, it should be noted that the tracking device 210 could comprise an additional shape sensing means 212, which could extend along a length of the elongated flexible body 200 and could be fixedly coupled to a known reference point.

Generally, the tracking device 210 can be fixed to the elongated flexible body 200 at a known location and can be fixed such that the tracking device 210 does not substantially move relative to the elongated flexible body 200. As the tracking device 210 can be fixed to a portion of the elongated flexible body 200, the tracking device 210 can provide a location and/or orientation of the portion of the elongated flexible body 200 in the patient space. As will be discussed, the position (location and/or orientation) of the portion of the elongated flexible body 200 determined from the tracking device 210 can be used in combination with data from the shape sensing means 212 to determine a configuration of the elongated flexible body 200 within the anatomical structure substantially in real-time.

In one example, as shown in FIG. 2, the tracking device 210 can be fixed to the proximal end 202. With the tracking device 210 fixed to the proximal end 202, the tracking device 210 can be observed external to the patient 12, and thus, a variety of tracking devices 210 could be employed with the elongated flexible body 200, such as the optical tracking device 58b or the electromagnetic tracking device 58a. Alternatively, if the tracking device 210 is coupled to the proximal end 202, then the tracking device 210 could comprise a fixture having a known position, and a portion of the elongated flexible body 200 could be held within the fixture. Typically, if the tracking device 210 is coupled to the proximal end 202, the tracking device 210 can be coupled to the exterior surface 206 of the elongated flexible body 200. However, if the tracking device 210 comprises an electromagnetic tracking device 58a, then the tracking device 210 could be coupled to the interior surface 208, or could be secured between one or more layers that comprise the exterior surface 206.

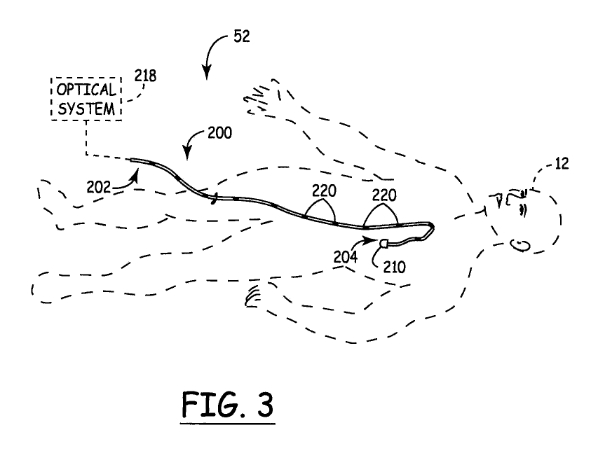

In one example, as shown in FIG. 3, the tracking device 210 can be fixed to the distal end 204. By fixing the tracking device 210 to the distal end 204, the tracking device 210 may not interfere with the manipulation of the elongated flexible body 200 by the user 39, and may improve accuracy in the computation of the location of the distal end 204 within the anatomical structure. With the tracking device 210 fixed to the distal end 204, however, the tracking device 210 generally cannot be observed outside of the patient 12. Thus, if the tracking device 210 is fixed to the distal end 204, the tracking device 210 can comprise an electromagnetic tracking sensor 58a, and/or electrodes responsive to an position sensing unit such as the LocaLisa® Intracardiac Navigation System, provided by Medtronic, Inc., for example. Generally, if the tracking device 210 comprises an electromagnetic tracking device 58a, then the tracking device 210 can be coupled to the interior surface 208, or could be secured between one or more layers that comprise the exterior surface 206.

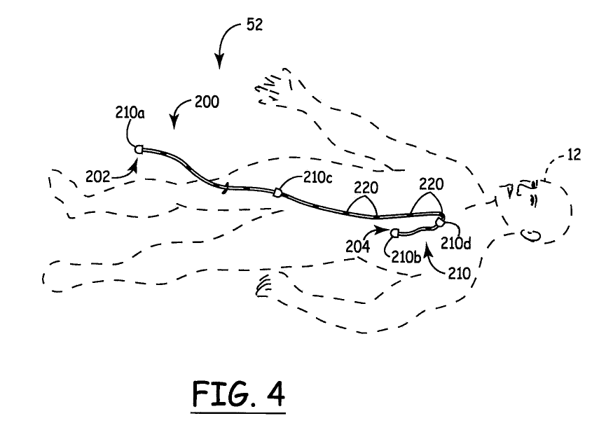

In one example, as illustrated in FIG. 4, the tracking device 210 can comprise at least two or a plurality of tracking devices 210. For example, the plurality of tracking devices 210 can include tracking devices 210a, 210b, 210c and 210d. The tracking device 210a can be coupled to the proximal end 202, and the tracking device 210b can be coupled to the distal end 204. The tracking devices 210c and 210d can be optional, and if employed, can be positioned between the proximal end 202 and the distal end 204. The use of the plurality of tracking devices 210 can ensure that that position of the distal end 204 within the anatomical structure matches the position of the distal end 204 as calculated by the control module 101 using the data from the tracking device 210a and the data from the shape sensing means 212, as will be discussed.

In addition, the use of the plurality of tracking devices 210 can ensure that the plurality of tracking devices 210 and the shape sensing means 212 are working properly. In this regard, if the position of the distal end 204 as determined by the shape sensing means 212 and the tracking device 210a does not correlate with the position of the distal end 210b, then the control module 101 can flag an error to notify the user 39 to service the elongated flexible body 200. Further, if the position of the portion of the elongated flexible body 200 coupled to the tracking device 210c does not correlate with the position of the portion of the elongated flexible body 200 determined from the tracking device 210a and the shape sensing means 212, then the control module 101 can also flag an error to notify the user to service the elongated flexible body 200.

It should also be noted that the tracking device 210 could also comprise at least one or a plurality of objects that are responsive to the imaging device 14 to generate positional data, such as one or more radio-opaque markers. Further, if the tracking devices 210 are radio-opaque markers, then the imaging device 14 can be used to track the position of the portion of the elongated flexible body 200 coupled to the tracking device 210. If the tracking device 210 comprises a radio-opaque marker, then the tracking device 210 can be coupled to the interior surface 208, or could be secured between one or more layers that comprise the exterior surface 206. In addition, the radio-opaque markers could be placed on an exterior surface 206 of the elongated flexible body 200.

With continued reference to FIGS. 2-4, the shape sensing means 212 can be used to determine a shape of the elongated flexible body 200 within the anatomical structure. In one example, as illustrated in FIG. 2A, the shape sensing means 212 can comprise at least one or a plurality of optical fibers 216 and an optical system 218. For example, the optical fibers 216 and the optical system 218 can comprise the optical fiber and optical system disclosed in U.S. Patent Pub. No. 2006/0013523, entitled “Fiber Optic Position and Shape Sensing Device and Method Relating Thereto,” hereby incorporated by reference, or the Distributed Sensing System™, commercially available from Luna Innovations Inc. of Blacksburg, Va., the optical fibers 216 and the optical system 218 will not be discussed in great detail herein.

Briefly, however, in one example, as illustrated in FIG. 2A, the optical fiber(s) 216 can be coupled to the interior surface 208, or could be secured between one or more layers that comprise the exterior surface 206 of the elongated flexible body 200, such as by extrusion. In one example, the optical fiber(s) 216 can comprise a single optical fiber 216 with a multi-core construction, which is described in more detail in U.S. Patent Pub. No. 2006/0013523, entitled “Fiber Optic Position and Shape Sensing Device and Method Relating Thereto,” and incorporated by reference herein in its entirety.

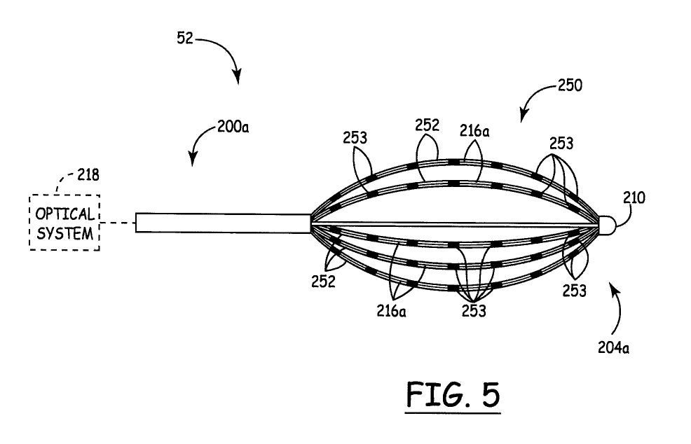

In one example, as illustrated in FIG. 5, with similar reference numerals corresponding to similar features, in the case of an elongated flexible body that includes a expandable portion, such as a balloon or basket catheter 200a, the optical fiber(s) 216a (shown schematically as a line for the sake of clarity) can be configured to expand along with the elongated flexible body 200a. For example, the basket catheter 200a can have a basket portion 250 adjacent to a distal end 204a, and the optical fiber(s) 216a can be configured to expand or contract with one or more spines 252 of the basket portion 250. It should be understood that the basket catheter 200a illustrated herein is merely exemplary, and any suitable basket catheter could employ the optical fiber(s) 216a, such as the Constellation™ sold by Boston Scientific, Inc. of Nantick, Mass.

In one example, each spine 252 includes a corresponding optical fiber 216a, and the distal end 204a can also include a tracking device 210. As each spine 252 includes a corresponding optical fiber 216a, the position and shape of each spine 252 can be determined, and thus, the position of at least one electrode 253 associated with the spine 252 can be determined without requiring the spine 252 to have a rigid fixed shape or without requiring the use of a plurality of tracking devices. It should be further noted that the basket catheter 250a can comprise any suitable basket catheter having any desired number of electrodes 253, and thus, for the sake of clarity, the basket catheter 250a is illustrated herein with a select number of electrodes 253.

Thus, the use of optical fibers 216a with each spine 252 can enable the use of dynamic and flexible spines 252, which can provide the user with additional freedom in treating the patient 12, such as in performing an ablation procedure. For example, as a position of the electrode 253 can be determined from the shape of the spines 252 and the tracking of the tracking device 210, the user 39 may use the navigation system 10 to plan a procedure on the anatomy, such as an ablation procedure. Given the position of the electrode 253 of each of the spines 252, the user 39 can more accurately determine a location of an arrhythmia, and can more precisely plan to treat the arrhythmia for example, by returning to a location identified by one of the electrodes 253 to perform an ablation procedure. Moreover, the use of a tracking device 210 at the distal end 204a can increase the accuracy of the position and shape obtained by the optical fibers 216a.

Each optical fiber 216 can include a plurality of strain sensors, such as fiber Bragg gratings 220 (schematically illustrated for the sake of clarity in FIGS. 2-4). The fiber Bragg gratings 220 can be formed on the optical fiber 216 such that any strain induced on the optical fiber 216 can be detected by the optical system 218. With regard to FIG. 5, the fiber Bragg gratings 220 (not specifically shown for clarity) can be positioned on the optical fiber(s) 216a such that a location of each of the electrodes 253 on each of the spines 252 can be determined from the strain data. The optical system 218 can use any suitable means to read the fiber Bragg gratings 220, such as optical frequency-domain reflectometry, wavelength division multiplexing, optical time-domain reflectometry, etc. Based on the data obtained from the optical system 218, the control module 101 can determine the shape of the elongated flexible body 200 within the anatomical structure.

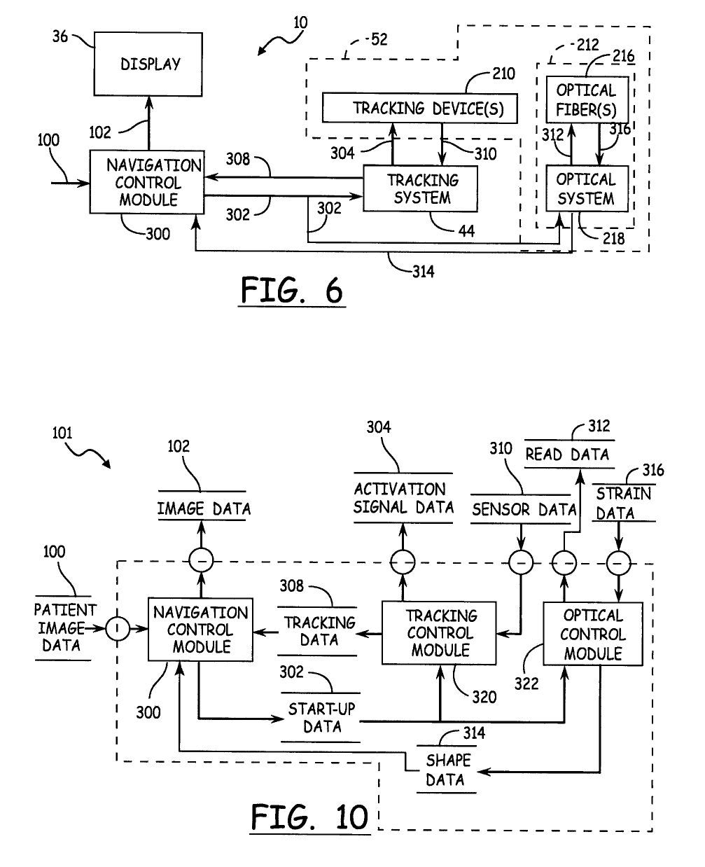

With reference now to FIG. 6, a simplified block diagram schematically illustrates an exemplary navigation system 10 for implementing the control module 101. The navigation system 10 can include the tracking system 44, the instrument 52, a navigation control module 300 and the display 36. The instrument 52 can include the tracking device(s) 210 and the shape sensing means 212, which can include the optical fiber(s) 216 and the optical system 218.

The tracking system 44 can comprise the electromagnetic tracking system 44, the optical tracking system 44b, or any other suitable tracking system, such as a position sensing unit, and will generally be referred to as the tracking system 44. The tracking system 44 can receive start-up data 302 from the navigation control module 300. In the case of an electromagnetic tracking system 44, based on the start-up data 302, the tracking system 44 can set activation signal data 304 that can activate the coil arrays 46, 47 to generate an electromagnetic field to which the tracking device(s) 210 coupled to the instrument 52 can respond. The tracking system 44 can also set tracking data 308 for the navigation control module 300, as will be discussed. The tracking data 308 can include data regarding the coordinate position (location and orientation) of the tracking device(s) 210 coupled to the instrument 52 in the patient space as computed from data received from the tracking device(s) 210 or sensor data 310.

When the tracking device(s) 210 are activated, the tracking device(s) 210 can transmit sensor data 310 indicative of a position of the tracking device 210 in the patient space to the tracking system 44. Based on the sensor data 310 received by the tracking system 44, the tracking system 44 can generate and set the tracking data 308 for the navigation control module 300.

The optical system 218 can also receive start-up data 302 from the navigation control module 300. Based on the start-up data 302, the optical system 218 can set read data 312 for the optical fiber(s) 216, which can read the fiber Bragg gratings 220 on each optical fiber 216. The optical system 218 can also set shape data 314 for the navigation control module 300, as will be discussed. The shape data 314 can include data regarding the shape of the instrument 52 in the patient space as computed from data received from the optical fiber(s) 216 or strain data 316.

When the optical fiber(s) 216 are read, any strain on the optical fiber(s) 216 can be read by the optical system 218 as strain data 316, which can be indicative of a shape of the instrument 52 in the patient space. Based on the strain data 316 received by the optical system 218, the optical system 218 can generate and set the shape data 314 for the navigation control module 300.

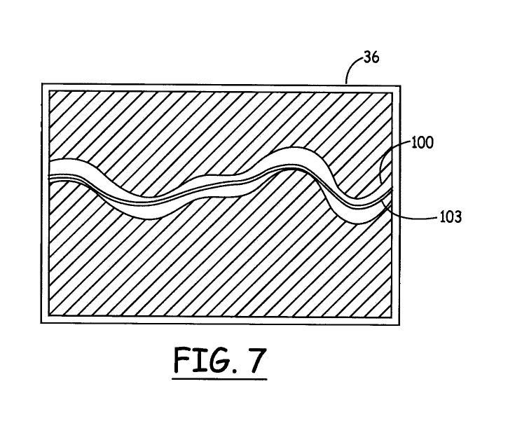

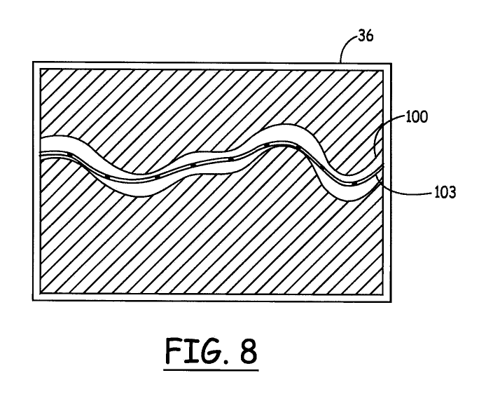

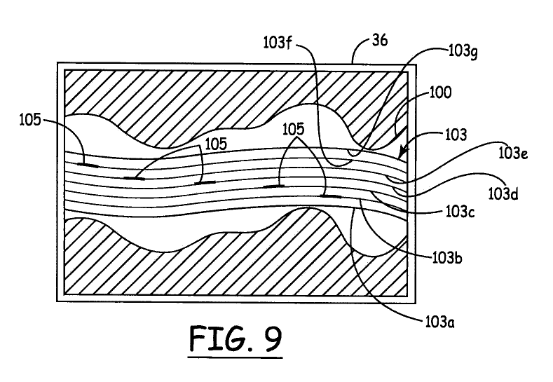

The navigation control module 300 can receive the tracking data 308 from the tracking system 44 and the shape data 314 from the optical system 218 as input. The navigation control module 300 can also receive patient image data 100 as input. The patient image data 100 can comprise images of the anatomical structure of the patient 12 obtained from a pre- or intra-operative imaging device, such as the images obtained by the imaging device 14. Based on the tracking data 308, the shape data 314 and the patient image data 100, the navigation control module 300 can generate image data 102 for display on the display 36. The image data 102 can comprise the patient image data 100 superimposed with an icon 103 of the instrument 52, with a substantially real-time indication of the position and a shape of the instrument 52 in patient space, as shown in FIG. 7. The image data 102 could also comprise a schematic illustration of the instrument 52 within the anatomical structure of the patient 12, etc. as shown in FIGS. 8 and 9.

For example, as shown in FIG. 7, the elongated flexible body 200 can be illustrated as the icon 103, and can be displayed on the display 36 with the patient data 100. The elongated flexible body 200 can be displayed relative to the patient data 100 at substantially the real-time position and shape of the elongated flexible body 200 within the anatomical structure of the patient 12. This can facilitate the navigation of the instrument 52, such as the elongated flexible body 200, by the user 39 within the anatomical structure of the patient 12.

In one example, as shown in FIG. 8, if the elongated flexible body 200 includes tracking device(s) 210 that comprise radio-opaque markers, then the icon 103 can include a graphical illustration of the instrument 52, along with the position and orientation of the radio-opaque markers as captured by the imaging device 14.

In one example, as shown in FIG. 9, if the elongated flexible body 200 comprises the basket catheter 200a that includes the spines 252, then the icon 103 can include a graphical illustration of each of the spines 252, numbered 103a-103g, which can include the position and shape of the spines 252 relative to the anatomical structure of the patient 12. In addition, the image data 102 can comprise icon(s) 105, which can indicate a position of the electrode 253 associated with each of the spines 252. This can enable the user 39 to ensure that the spines 252 are positioned as desired within the anatomical structure, and so each respective spine 252 or electrode 253 location can be subsequently recorded and returned to with the same or different instruments.

With reference now to FIG. 10, a dataflow diagram illustrates an exemplary control system that can be embedded within the control module 101. Various embodiments of the control system according to the present disclosure can include any number of sub-modules embedded within the control module 101. The sub-modules shown may be combined and/or further partitioned to similarly determine the position and shape of the instrument 52 within the patient space based on the signals generated by the tracking device(s) 210 and the shape sensing means 212. In various embodiments, the control module 101 includes the tracking system 44 that can implement a tracking control module 320, the optical system 218 that can implement an optical control module 322, and the workstation 34 that can implement the navigation control module 300. It should be noted, however, that the tracking control module 320, the optical control module 322 and the navigation control module 300 could be implemented on the workstation 34, if desired.

The tracking control module 320 can receive as input the start-up data 302 from the navigation control module 300 and sensor data 310 from the tracking device(s) 210. Upon receipt of the start-up data 302, the tracking control module 320 can output the activation signal data 304 for the tracking device(s) 210. Upon receipt of the sensor data 310, the tracking control module 320 can set the tracking data 308 for the navigation control module 300. As discussed, the tracking data 308 can include data regarding the coordinate positions (locations and orientations) of the instrument 52.

The optical control module 322 can receive as input the start-up data 302 from the navigation control module 300 and strain data 316 from the optical fiber(s) 216. Upon receipt of the start-up data 302, the optical control module 322 can output the read data 312 to the optical fiber(s) 216. Upon receipt of the strain data 316, the optical control module 322 can set the shape data 314 for the navigation control module 300. As discussed, the shape data 314 can include data regarding the shape of the instrument 52 in the patient space.

The navigation control module 300 can receive as input the tracking data 308, the shape data 314 and patient image data 100. Based on the tracking data 308 and the shape data 314, the navigation control module 300 can determine the appropriate patient image data 100 for display on the display 36, and can output both the tracking data 308, shape data 314 and the patient image data 100 as image data 102. Further, depending upon the number of tracking device(s) 210 employed, the navigation control module 300 can determine if the shape sensing means 212 is working properly, and can output a notification message to the display 36 if the tracking data 308 does not correspond with the shape data 314. In addition, the navigation control module 300 could override or correct the shape data 314 if the shape data 314 does not correspond with the tracking data 308, or could override or correct the tracking data 308 if the tracking data 308 does not correspond with the shape data 314, if desired.

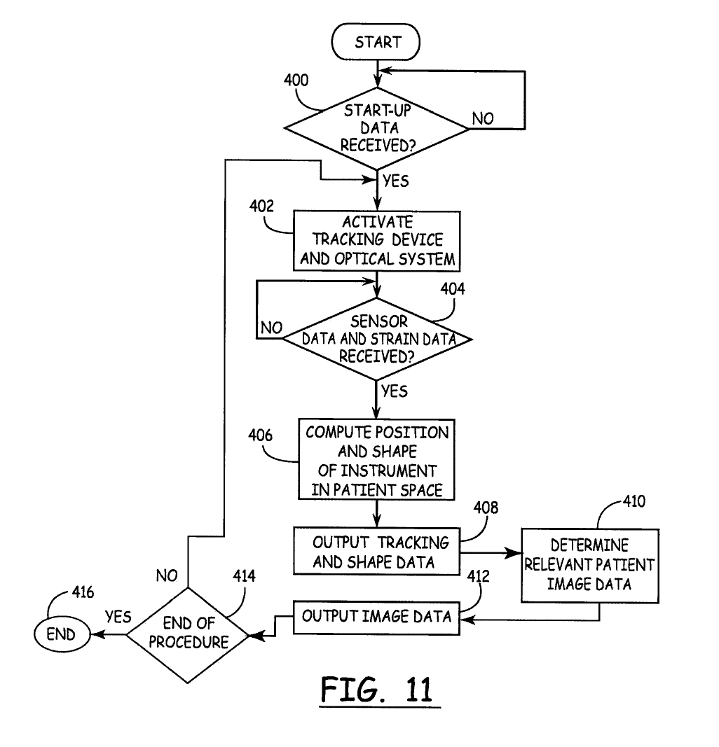

With reference now to FIG. 11, a flowchart diagram illustrates an exemplary method performed by the control module 101. At decision block 400, the method can determine if start-up data 302 has been received from the navigation control module 300. If no start-up data 302 has been received, then the method loops to decision block 400 until start-up data 302 is received. If start-up data 302 is received, then the method goes to block 402. At block 402, the tracking system 44 can generate the activation signal data 304 and the optical system 218 can generate the read data 312. Then, at decision block 404 the method can determine if the sensor data 310 and the strain data 316 have been received. If the sensor data 310 and strain data 316 have been received, then the method goes to block 406. Otherwise, the method loops to decision block 404 until the sensor data 310 and the strain data 316 are received.

At block 406, the method can compute the position and shape of the instrument 52 in patient space based on the sensor data 310 and the strain data 316. In this regard, the sensor data 310 can provide a position of the tracking device(s) 210 in patient space, and the strain data 316 can provide a shape of the instrument 52 in the patient space based on the strain observed by the optical fiber(s) 216. At block 408, the method can output the tracking data 308 and the shape data 314. At block 410, the method determines the relevant patient image data 100 for display on the display 36 based on the tracking data 308 and the shape data 314. Then, at block 412, the method can output the image data 102 that includes the icon 103 of the instrument 52 superimposed on the patient image data 100 based on the patient image data 100, the tracking data 308 and the shape data 314. At decision block 414, the method can determine if the surgical procedure has ended. If the surgical procedure has ended, then the method can end at 416. Otherwise, the method can loop to block 402.

Therefore, the instrument 52 of the present disclosure, for example, the elongated flexible body 200, can provide a user, such as a surgeon, with an accurate representation of the position and shape of the instrument 52 within the patient space during the surgical procedure. In this regard, the use of a shape sensing means 212 along with the tracking device(s) 210 can enable an accurate depiction of the position and shape of an elongated instrument, such as the elongated flexible body 200, within the anatomical structure of the patient 12. In addition, if multiple tracking devices 210 are employed with the shape sensing means 212, then the navigation system 10 can update the user regarding the accuracy of the instrument 52. Thus, if the elongated flexible body 200 or optical fiber(s) 216 are dropped, bent or otherwise damaged during the procedure, the use of multiple tracking devices 210 at a known location on the elongated flexible body 200 can enable the navigation system 10 to verify the accuracy of the instrument 52 throughout the surgical procedure.

While specific examples have been described in the specification and illustrated in the drawings, it will be understood by those of ordinary skill in the art that various changes may be made and equivalents may be substituted for elements thereof without departing from the scope of the present disclosure as defined in the claims. Furthermore, the combination of features, elements and/or functions between various examples is expressly contemplated herein so that one of ordinary skill in the art would appreciate from this disclosure that features, elements and/or functions of one example may be incorporated into another example as appropriate, unless described otherwise, above. Moreover, many modifications may be made to adapt a particular situation or material to the teachings of the present disclosure without departing from the essential scope thereof. Therefore, it is intended that the present disclosure not be limited to the particular examples illustrated by the drawings and described in the specification as the best mode presently contemplated for carrying out this disclosure, but that the scope of the present disclosure will include any embodiments falling within the foregoing description and the appended claims.

For example, while the instrument 52, such as the elongated flexible body 200 has been described as including a tracking device 210, those of skill in the art will appreciate that the present disclosure, in its broadest aspects, may be constructed somewhat differently. In this regard, the elongated flexible body 200 could only include the shape sensing means 212. If the elongated flexible body 200 included only the shape sensing means 212, then in order to register the position of the elongated flexible body 200 relative to the anatomical structure, the entry position of the elongated flexible body 200 could be marked on the patient 12, with a radio-opaque marker for example. Then, the imaging device 14 can acquire an image of the patient 12 that includes the marked entry position. If gating is desired, multiple images of the patient 12 can be acquired by the imaging device 14. As the entry position is known to the navigation system 10, via the acquired image, and the length of the elongated flexible body 200 is known, the shape and position of the elongated flexible body 200 within the anatomical structure can be determined by the control module 101, and outputted at image data 102 substantially in real-time.

What is claimed is:

1. A system for tracking an instrument relative to an anatomical structure comprising:
at least one tracking device coupled to the instrument;
a shape sensor coupled to the instrument that determines a shape of the instrument;
a tracking system that tracks a position of the at least one tracking device relative to the anatomical structure; and
a navigation system that determines a position and shape of the instrument relative to the anatomical structure based on the position of the at least one tracking device determined by the tracking system and the shape of the instrument as sensed by the shape sensor.

2. The system of claim 1, further comprising:
an imaging device that is operable to acquire an image of the anatomical structure.

3. The system of claim 2, further comprising:
a display that displays the image of the anatomical structure superimposed with an icon of the instrument at a location that corresponds to the position of the instrument relative to the anatomical structure, and displays the shape of the instrument.

4. The system of claim 3, wherein the instrument is an elongated instrument, and includes a proximal end and a distal end, and the shape sensor is able to determine the shape of the instrument from a region proximate the distal end to a region proximate the proximal end.

5. The system of claim 4, wherein the instrument is selected from the group comprising:
catheters, basket catheters, balloon catheters, leads, guidewires, sheaths, endoscopes, ablation catheters, arthroscopic systems, orthopedic implants, spinal implants, deep-brain stimulator (DBS) probes, drug delivery systems, mapping catheters, drill bits, stylets, trocars, screws or combinations thereof.

6. The system of claim 4, wherein the at least one tracking device comprises a plurality of tracking devices, with at least one of the plurality of tracking devices coupled to the proximal end of the instrument, at least one of the plurality of tracking devices coupled to the distal end of the instrument, and at least one of the plurality of tracking devices coupled between the proximal end and the distal end of the instrument, and the shape sensor is located proximate to the at least one tracking device coupled to the proximal end, the at least one tracking device coupled to the distal end and the at least one tracking device coupled between the proximal end and the distal end.

7. The system of claim 6, wherein the navigation system outputs a notification message to the display if a position of the distal end of the instrument determined from the tracking of the at least one tracking device at the distal end of the instrument does not substantially correspond to a position of the distal end of the instrument determined from the shape sensor.

8. The system of claim 6, wherein the at least one tracking device comprises at least one optical tracking device to track at least one degree of freedom information.

9. The system of claim 6, wherein the at least one tracking device comprises at least one electromagnetic tracking device selected from the group including: an electromagnetic receiver tracking device, an electromagnetic transmitter tracking device and combinations thereof.

10. The system of claim 1, wherein the shape sensor further comprises at least one optical fiber that is coupled to the instrument.

11. The system of claim 10, wherein the at least one optical fiber includes a plurality of fiber Bragg gratings.

12. The system of claim 10, wherein the instrument comprises a basket catheter having a plurality of spines, with each of the spines coupled to an optical fiber to enable the shape sensor to determine a shape of each of the plurality of splines.

13. The system of claim 12, wherein each of the plurality of spines includes at least one electrode, and the navigation system determines a position of the at least one electrode of each of the plurality of spines based on the shape of each of the spines determined from the shape sensor.

14. The system of claim 13, wherein the at least one tracking sensor is coupled adjacent to the plurality of spines to enable the navigation system to determine a position of the plurality of spines, and the position of the plurality of spines and the position of the at least one electrode of each of the plurality of spines is used to plan a procedure on the anatomy.

15. The system of claim 14, wherein the procedure is an ablation.

16. The system of claim 15, wherein the ablation procedure is performed with a separate tool or instrument than the basket catheter.

17. The system of claim 1, wherein the at least one tracking device comprises at least one radio-opaque marker, and the tracking system comprises an imaging device operable to image the anatomical structure to track the position of the at least one radio-opaque marker relative to the anatomical structure.

18. A method for tracking an instrument relative to an anatomical structure comprising:
positioning at least one tracking device on the instrument;
coupling a shape sensor to the instrument;
tracking the at least one tracking device relative to the anatomical structure;
sensing a shape of the instrument;
determining, based on the tracking of the at least one tracking device and the shape of the instrument, a position of instrument relative to the anatomical structure; and
displaying the position of the instrument and the shape of the instrument relative to the anatomical structure as an icon superimposed on an image of the anatomical structure.

19. The method of claim 18, further comprising:
acquiring an image of the anatomical structure with an imaging device selected from at least one of a fluoroscopy device, an O-arm device, a bi-plane fluoroscopy device, an ultrasound device, a computed tomography (CT) device, a multi-slice computed tomography (MSCT) device, a magnetic resonance imaging (MRI) device, a high frequency ultrasound (HFU) device, a positron emission tomography (PET) device, an optical coherence tomography (OCT) device, an intra-vascular ultrasound (IVUS) device, an intra-operative CT device, an intra-operative MRI device or combinations thereof.

20. The method of claim 18, wherein sensing a shape of the instrument further comprises:
determining a strain on at least one optical fiber coupled to the instrument.

21. The method of claim 18, wherein tracking at least one tracking device further comprises:
tracking a tracking device coupled to a proximal end of the instrument;
tracking a tracking device coupled to a distal end of the instrument; or combinations thereof.

22. The method of claim 21, further comprising:
determining, based on the tracking of the tracking device coupled to the proximal end and the shape of the instrument, a position of the instrument relative to the anatomical structure;
determining, based on the tracking of the tracking device coupled to the distal end of the instrument a position of the instrument relative to the anatomical structure; and
displaying notification data if the position of the instrument determined by the tracking of the tracking device coupled to the proximal end and the shape of the instrument does not substantially correspond to the position of the instrument determined by the tracking of the tracking device coupled to the distal end of the instrument.

23. A system for tracking an instrument relative to an anatomical structure comprising:
an elongated flexible body having a proximal end and a distal end for insertion into the anatomical structure;
at least one tracking device coupled to the proximal end, the distal end, a portion of the elongated flexible body between the proximal end and the distal end or combinations thereof;
at least one optical fiber coupled to the elongated flexible body that includes a plurality of strain sensors;
a tracking system that tracks a position of the tracking device relative to the anatomical structure;
an optical system that reads the plurality of strain sensors on the at least one optical fiber;
a navigation system that determines a position of the elongated flexible body based on the tracking of the first tracking device and a shape of the elongated flexible body based on the reading of the plurality of strain sensors; and
a display that displays an image of the anatomical structure with the position and shape of the elongated flexible body superimposed on the anatomical structure.

24. The system of claim 23, wherein the position and shape of the elongated flexible body is determined in response to a physiological event.

25. The system of claim 24, wherein the image of the anatomical structure is acquired in response to the physiological event, and the display displays an icon of the position and shape of the elongated flexible body at the physiological event superimposed over the image of the anatomical structure acquired at the physiological event.
